(19) United States
(12) Patent Application Publication
(10) Pub. No.: US 2004/0094164 A1
(43) Pub. Date: May 20, 2004

(54) Condom for prolonging penile erections

(76) Inventors: Aaron Feingold, Morganville, NJ (US); Jean-Marc Zimmerman, Westfield, NJ (US)

Correspondence Address: Jean-Marc Zimmerman, 226 St. Paul Street, Westfield, NJ, 07090, US

(21) Appl. No.: 10/703,721

(22) Filed: Nov. 8, 2003

Related U.S. Application Data
(60) Provisional application No. 60/427,757, filed on Nov. 20, 2002.

(51) Int. Cl. A61F 6/04

(52) U.S. Cl. 128/844

(57) ABSTRACT

An improved condom for prolonging erections by means of a selectively adjustable constriction band fabricated as an integral element of the condom, wherein the band constricts blood flow through the penis to prolong the erection. The degree of constriction provided by the constriction band can be selectively adjusted by the person wearing the condom. The constriction band can be fabricated to include an O-ring and ball-locking element for selectively adjusting the degree of constriction around the penis. In an alternative embodiment, the constriction band can also be fabricated to include a stent that can be removed through an opening in the band to cause the ring to constrict around the penis. In another alternative embodiment, the constriction band can be fabricated as a selectively detachable element that can be detached from the condom when an individual wearing the condom so desires.

### RELATED APPLICATIONS

This Application claims the priority of Provisional Patent Application Serial No. 06/427,757 filed by Aaron Feingold and Jean-Marc Zimmerman on Nov. 8, 2002.

### FIELD OF INVENTION

The present invention pertains to condoms and more particularly to condoms that prolong erections.

### BACKGROUND OF INVENTION

Condoms are widely used for birth control purposes as well as to prevent the spread of sexually transmitted diseases. Conventional condoms, as shown in FIG. 1, are typically fabricated as a tube-shaped apparatus 10 having a closed tip reservoir at one end 12 for receiving bodily fluids such as ejaculated semen and an oppositely-disposed open end 14 through which a penis is inserted into the condom. Condoms are typically sold as a rolled-up apparatus in an individual, single-use package with ends 12 and 14 in planar alignment to one another. A condom is typically placed on an erect penis by inserting the tip of the erect penis through open end 14 so that it rests against end 12 and then unrolling the rolled-up condom over the erect penis from the tip of the penis towards the base of the penis. Conventional condoms are typically fabricated from latex or sheepskin, and can be of varying size, sensitivity, elasticity and color. No conventional condoms sold on the market are able to help prolong erections.

Penile support or constriction rings are used to aid men who have difficulty maintaining erections or can only achieve partial erections. Conventional support rings are worn around the penis to constrict the blood flow through the penis to promote firmer, longer lasting erections. Such rings are typically made of silicon or similar materials and come in a variety of sizes. A problem commonly encountered with conventional support rings is that men wearing such rings often lose their erections due to venous return blood flow from the penis when they attempt to place a condom on their penis.

It is an object of the present invention to overcome the drawbacks associated with conventional condoms and support rings by combining them to make a new apparatus.

### SUMMARY OF THE INVENTION

An improved condom that is able to prolong erections by means of a selectively adjustable constriction band fabricated as an integral element of the condom. The degree of constriction provided by the constriction band can be selectively adjusted by the person wearing the condom. In an alternative embodiment, the constriction band can be fabricated as a selectively detachable element that can be detached from the condom when an individual wearing the condom so desires. In still another alternative embodiment, the condom includes a semi-rigid structure around the open end of the condom to aid individuals in placing the condom on an erect penis.

### BRIEF DESCRIPTION OF THE DRAWINGS

FIG. 1 shows a conventional condom.

FIG. 2 shows an exemplary embodiment of an improved condom according to the present invention.

FIG. 3 shows another exemplary embodiment of an improved condom according to the present invention.

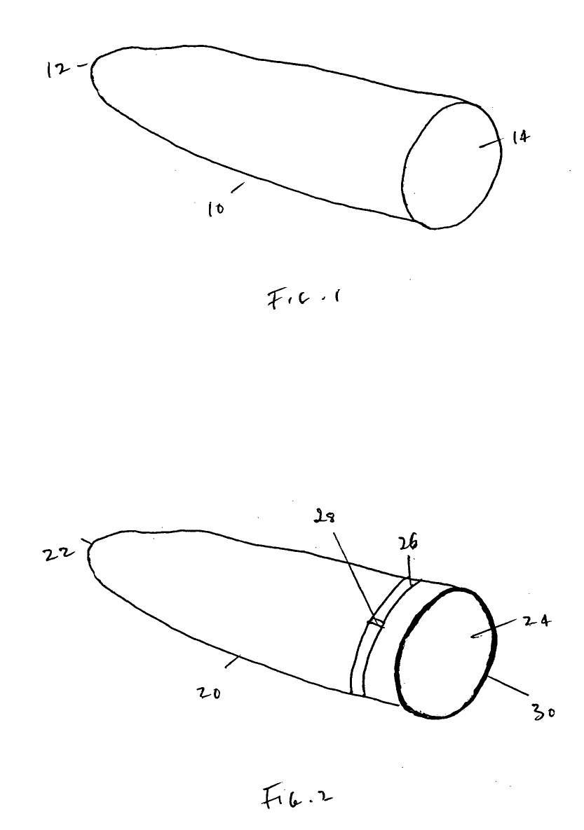

### DETAILED DESCRIPTION OF THE INVENTION

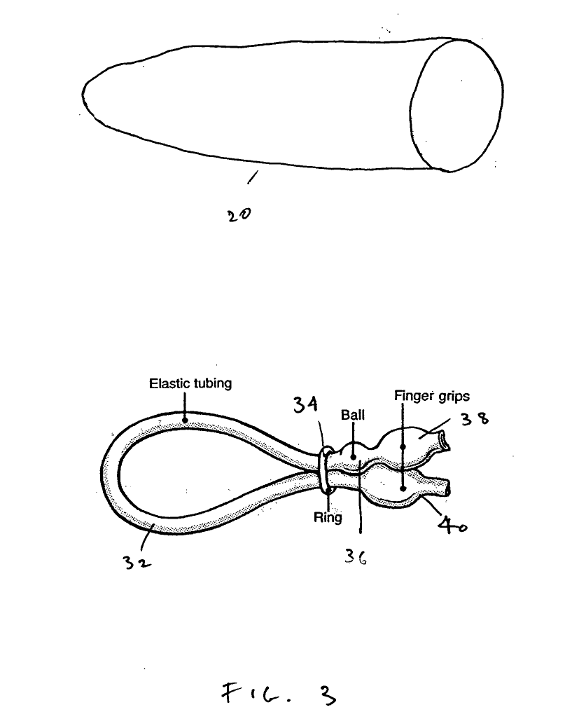

FIG. 2 shows an exemplary embodiment of the improved condom 20 according to the present invention. Condom 20 includes a closed tip reservoir at one end 22 for receiving bodily fluids such as ejaculated semen and an oppositely-disposed open end 24 through which a penis is inserted into the condom. Condom 20 also includes a selectively adjustable constriction band 26 that serves the same purpose as a conventional support ring. Constriction band 26 is positioned around a circumferential portion of condom 20. Constriction band 26 enables a man wearing condom 20 to selectively adjust the degree of constriction around their penis to help them attain firmer erections of longer duration.

Constriction band 26 can be fabricated as part of condom 20. Alternatively, constriction band 26 can be fabricated as an element fastened to the exterior of condom 20. Constriction band 26 can be fabricated from the same material as that used to fabricate condom 20, or alternatively it can be fabricated from a different material. Constriction band 26 is ideally fabricated from an expandable and contractible material. Constriction band 26 can be positioned anywhere between end 22 and open end 24, although ideally it is positioned proximate to open end 24.

Constriction band 26 enables a man wearing condom 20 to tighten the condom to as great an extent as desired. Constriction band 26 includes a moveable end 28 that can be moved along and adhered to different points along band 26 to tighten the degree of constriction provided by band 26. End 28 is moved by pulling it up off and away from a first position along band 26 and then moving it to a second position along band 26. End 28 is adhered to band 26 by an adhesive means that is applied to the bottom portion of end 28 that rests on band 26, and/or is applied to the entire circumference of or selected points along band 26. Constriction band 26 can be loosened by pulling up end 28 from the second position to which it has been adhered.

In an another alternative embodiment of the present invention, constriction band 26 can be fabricated as an element that is selectively detachable from condom 20 so that when a man seeks to loosen the degree of constriction around their penis he can detachably pull and detach band 26 from condom 20 without ripping or otherwise damaging condom 20.

In another alternative embodiment of the present invention, constriction band 26 can be fabricated to include 1) a stent that maintains an expanded shape of band 26, and 2) an opening, wherein removing the stent by pulling it out through the opening causes constriction band 26 to contract and tighten around the penis.

In still another alternative embodiment of the present invention, condom 20 includes a semi-rigid or rigid collar-like structure 30 that is akin to a shoe-horn for aiding an individual to place condom 20 on their penis without losing their erection. Structure 30 is positioned proximate to open end 24, and can be fabricated as part of open end 24.

Condom 20 can be fabricated from various materials, and can be of varying size, sensitivity, elasticity and color. Constrictive band 26, end 28 and structure 30 can each be fabricated from the same material or they can be fabricated from different materials.

FIG. 3 shows another alternative embodiment of the present invention in which constriction band 26 is comprised of an elastic tubing 32, a ring 34, and a ball-locking element 36 having finger grips 38 and 40. In this embodiment, constriction band 26 is an adjustable loop around the circumference of condom 20, wherein an O-ring and ball-locking mechanism allows the constriction around the penis to be selectively tightened or loosened by slidably moving ring 34 along tubing 32.

In an alternative embodiment of condom 20, constriction band 26 can be fabricated as a malleable element that can be squeezed to apply constrictive pressure to a penis, and can be malleably deformed to lessen such pressure.

Numerous modifications to and alternative embodiments of the present invention will be apparent to those skilled in the art in view of the foregoing description. Accordingly, this description is to be construed as illustrative only and is for the purpose of teaching those skilled in the art the best mode of carrying out the invention. Details of the structure may be varied substantially without departing from the spirit of the invention and the exclusive use of all the modifications which come within the scope of the appended claims is reserved.

What we claim is:

1. A condom for prolonging penile erections, comprising:
a condom; and
a constriction band for selectively constricting blood flow through an erect penis to prolong the erection.

2. The condom of claim 1, wherein the constriction band is fabricated as an integral element of the condom.

3. The condom of claim 1, wherein the degree of constriction applied by the constriction band is selectively adjustable.

4. The condom of claim 1, wherein the constriction band is fabricated from a material identical to that from which the condom is fabricated.

5. The condom of claim 1, wherein the constriction band is fabricated from a material different from the condom.

6. The condom of claim 1, wherein the constriction band includes a moveable end that can be moved along and adhered to different points along the band to tighten the degree of constriction provided by the band.
